(19) United States
(12) Patent Application Publication
(10) Pub. No.: US 2023/0218889 A1
(43) Pub. Date: Jul. 13, 2023

(54) Methods and Systems for Optimizing Placement of a Nerve Stimulation Device

(71) Applicant: The Trustees of Columbia University in the City of New York, New York, NY (US)

(72) Inventors: Charles RODENKIRCH, New York, NY (US); Qi WANG, New York, NY (US)

(73) Assignee: The Trustees of Columbia University in the City of New York, New York, NY (US)

(21) Appl. No.: 18/121,899

(22) Filed: Mar. 15, 2023

Related U.S. Application Data
(63) Continuation of application No. PCT/US2021/050805, filed on Sep. 17, 2021.
(60) Provisional application No. 63/080,136, filed on Sep. 18, 2020.

(51) Int. Cl. A61N 1/04

(52) U.S. Cl. CPC A61N 1/0456; A61N 1/0476

(57) ABSTRACT

Methods and devices for positioning a nerve stimulation device on a subject are provided. Aspects are directed to methods of positioning a stimulation device on an anatomic target of a subject comprising positioning an alignment guide adjacent to at least a first alignment point associated with the anatomic target on the subject, wherein the alignment guide indicates a first target location on the anatomic target of the subject, and positioning the stimulation device at the first target location.

### CROSS REFERENCE TO RELATED APPLICATIONS

This Application is a continuation of International Application PCT/US2021/050805, filed Sep. 17, 2021, which claims priority to and the benefit of U.S. Provisional Application 63/080,136, filed Sep. 18, 2020, each of which is incorporated herein by reference in its entirety.

### BACKGROUND

The field of wearable devices for medical applications is rapidly expanding. These wearable devices typically modulate biological activity at a specific anatomic point of interest by delivering bursts of energy. Wearable devices can also be used to record biosignals. However, currently available wearable devices are typically placed on the body by the user without any guidance or feedback. Without accurate placement of the wearable device, the ability to optimally deliver energy or record biosignals is limited.

Many of these devices function optimally with, or will not function without, accurate placement of the device adjacent to a specific anatomic point of interest. An anatomical point of interest on or within the body is often a target for a device to either read or modulate biological activity (e.g., peripheral nerve stimulation or electromyography). For example, there a variety of techniques that allow for manipulation of the activity of nerves, muscle groups, blood vessels, or organs located underneath the skin by delivering bursts of energy (e.g., electrical, ultrasonic, vibrational, etc.) through the skin to these targets or systems that influence these targets. Variation in the parameters of this energy delivered (e.g., frequency, amplitude, duty-cycle, etc.) have been shown to allow for driving unique effects suggesting a large potential for expansion in the use of these devices for applications like bioelectronic medicine or performance enhancement. This energy is often targeted solely based on the position of the electrical stimulation device. Further, there are a variety of wearable devices which record biosignals, including electrodermal activity, electromagnetic fields or potentials, auditory signals, and physical deformations. The positioning of these devices relative to the source of the signals they are recording in many cases can affect their performance.

The present inventors have developed a bioelectronic device that delivers energy in a targeted fashion to activate a specific nerve. This technology could benefit from methods and systems of ensuring accurate placement over the target location. Specifically, recent work has shown that the locus coeruleus (LC), the sole source of norepinephrine (NE) to the forebrain, provides behavioral-state-relevant modulation of the neural coding in the early stage of the somatosensory pathway. Specifically, it was found that LC activation enhances thalamic feature selectivity via norepinephrine regulation of intrathalamic circuit dynamics. Modulation of sensory processing has many translational applications; however, the LC is a deep brainstem nucleus which prevents direct noninvasive activation with currently available techniques. However, peripheral nerve stimulation techniques provide a pathway for treatment, due to their ability to readily activate downstream neuromodulatory systems with minimal invasiveness and reduced side effects. Previous research has shown that vagus nerve stimulation (VNS) activates the LC. Further, VNS has been approved by the FDA (U.S. Food and Drug Administration) for use in treatment of epilepsy and tinnitus in humans, and has been proposed as a treatment for a wide variety of neurodisorders including depression, autism, stroke-induced damage, and PTSD (post-traumatic stress disorder). Recently, techniques allowing for non-invasive transcutaneous VNS have been developed and commercially implemented. VNS has been shown to activate neuromodulatory networks, including the locus-coeruleus-norepinephrine (LC-NE) system.

Previous work has focused on using the VNS to facilitate the neuroplasticity of brain circuits, likely through activation of neuromodulatory pathways. These VNS-induced neuroplasticity-driven changes can persist over long timescales.

Locus Coeruleus (LC) activation improves feature selectivity in the ventral posteromedial nucleus (VPm), effectively increasing the sensory-stimulus related information transmitted by thalamic relay neurons to the cortex resulting in improved perception of details of sensory stimuli. Vagus nerve stimulation (VNS) can be used to increase LC activity. VNS has been studied as a therapy to treat neurological disorders including epilepsy, depression, stroke, and tinnitus. LC activation has been correlated with pupil diameter.

When sensory information enters the brain, it is encoded as a neural signal. The encoded sensory information is then processed through multiple brain regions prior to perception. This processing of sensory information is imperfect, introducing noise that degrades the accuracy of the resulting perception. Therefore, perceptual acuity is dependent on the quality of sensory processing.

Accurate perception of details in tactile, auditory, and visual stimuli is useful for performing tasks correctly and safely. Once sensory information is encoded as neural activity, it is processed through multiple brain regions (i.e., thalamus, cortex) before perception occurs. Therefore, perceptual acuity is dependent upon high-fidelity, accurate processing of sensory stimuli by the brain (i.e., sensory processing). Accuracy of perception exerts a heavy influence on an individual's ability to complete workplace tasks, compete at sports, or even enjoy hobbies. Unfortunately, sensory loss is all too common. For example, one study found 94 percent of adults over 57 years of age had a deficiency in at least one sensory modality. This suggests that, in the United States, roughly 64 million suffer from some form of age-related sensory loss. As the elderly population grows, the population suffering from age-related sensory loss will increase, stressing current facilities that are not well designed to accommodate individuals with impaired senses. However, elderly individuals are not the only ones at risk of sensory loss. In addition to aging, traumatic brain injury (TBI) and various neurological disorders can also degrade sensory acuity. Finally, even individuals with normally accurate perception can occasionally suffer from impaired senses. This is because there are multiple commonly occurring factors, such as fatigue and inattention, that can degrade the sensory acuity of individuals with usually healthy senses.

Our reliance on our senses makes sensory loss highly disruptive to quality of life. Sensory loss is well-known to be isolating and can have devasting effects on mental health. Impaired senses in the elderly are especially damaging as they can interfere with their ability to live independently. For example, compromised sense of touch often leads to difficulty buttoning shirts or grasping objects needed to complete personal hygiene tasks. Degraded visual and auditory senses result in communication breakdown and stress important support relationships. The combined effects of sensory loss often result in depression, anxiety, and withdrawal from social situations. Finally, sensory loss is associated with increased risk of accidents, such as falls, that can have life threatening consequences. Even temporarily impaired senses in otherwise healthy individuals, which can occur due to fatigue or inattention, can cause significant negative effects. For example, sensory misperceptions arising from degraded sensory acuity can result in costly human error for military service members or workers who operate heavy machinery. Further, for individuals competing at sports or e-sports where peak performance is key, inaccurate perception can cause incorrect decisions and failure.

There is currently a dearth of available methods for improving sensory processing and those that do exist have many drawbacks. Stimulants improve sensory processing but cause cardiac damage, insomnia, anxiety, and addiction. Various nootropics brands make often unverified claims their supplements improve brain function. However, nootropics are largely ineffective and occasionally dangerous due lack of proper testing. For example, one research group found that after they published minimal preclinical research suggesting a compound might improve cognitive function, a nootropics company begun marketing the compound without any tests of long-term toxicity. Consumers' willingness to potentially risk their health by consuming research grade compounds without clinical testing highlights an unfulfilled need for technology that can improve sensory ability. Finally, as both stimulants and nootropics are taken orally, their effect has a delayed onset (30 to 60 minutes from ingestion) and cannot be turned off if desired. Taken together, these observations make it clear there is an unmet clinical need for bioelectronic technology that can improve sensory processing on-demand without risk of addiction, cardiac damage, or insomnia.

What is needed are methods of placement and positioning of devices to improve delivery and perception of sensory information to, for example, enhance sensory perception and treat sensory components of neurological disorders.

### SUMMARY OF THE INVENTION

In some instances, a first method of positioning a stimulation device on an anatomic target of a subject is provided. The first method comprises positioning an alignment guide adjacent to at least a first alignment point associated with the anatomic target on the subject, wherein the alignment guide indicates a first target location on the anatomic target of the subject, and positioning the stimulation device at the first target location.

In some instances, a second method of positioning a stimulation device on an anatomic target of a subject is provided. The second method comprises scanning the anatomic target on a body of a subject with an imaging device to obtain a target location scan for a target location, comparing the target location scan to one or more known scans of the anatomic target, generating an alignment guide for the anatomic target based on a comparison of the target location scan to the one or more known scans, positioning the alignment guide at a first alignment point wherein the alignment guide indicates a first target location on the anatomic target, and positioning the stimulation device at the first target location.

In some instances, a third method of positioning a stimulation device on an anatomic target of a subject is provided. The third method comprises scanning an anatomic target on a body of the subject with an imaging device to obtain a target location scan, generating an alignment guide for the anatomic target based on the target location scan, positioning the alignment guide adjacent to at least a first alignment point, wherein the alignment guide indicates a first target location on the anatomic target of the subject, and positioning the stimulation device at the first target location.

In some instances, a fourth method of applying stimulation to an anatomic target of a subject is provided. The fourth method comprises positioning an alignment guide adjacent to at least a first alignment point associated with the anatomic target on the subject wherein the alignment guide indicates a first target location on the anatomic target of the subject, positioning a stimulation device at the first target location, and applying stimulation to a location other than the first target location with the stimulation device.

Aspects described herein provide a system for applying stimulation to an anatomic target on a body of a subject comprising an alignment guide, wherein when the alignment guide is positioned at a first alignment point, the alignment guide indicates a first target location at the anatomic target; and a stimulation device for applying stimulation at the first target location.

### BRIEF DESCRIPTION OF DRAWINGS

FIG. 1 illustrates an exemplary method of positioning a stimulation device at a target location on a subject;

FIG. 2 illustrates an exemplary method of positioning a stimulation device at a target location on a subject using a scan of an anatomic target on a subject; and

FIG. 3 illustrates an exemplary alignment guide comprising an electrode grid and use of a pupil dilation and electrode stimulation to guide use of a stimulation device at a target location on a subject.

### DETAILED DESCRIPTION

All references cited herein, including, but not limited to patents and patent applications, are incorporated by reference in their entirety. Certain data disclosed herein was published after the earliest priority date of this application.

Although there is a rapidly growing field of wearable devices which read from or record from various specific locations on the body, there is a need for methods and systems that provide precise, repeatable, quick, and accurate placement by end users of these device, the majority of which will likely not have formal medical education. Such methods of and systems for placement and positioning of devices relative to anatomical landmarks or positions of interest can be used by untrained end users of these wearable devices.

The specific use of the device may dictate whether the device should be positioned close to, some intermediate position from, or far away from an anatomic target. Anatomic targets include, for example, nerves, muscles, organs, blood vessels, and other unique anatomical structures. One example of a technology that could benefit from this method of ensuring accurate placement is vagus nerve stimulation (VNS) through use of stimulation devices.

Proper positioning of stimulation devices permits more accurate collection of measured responses (e.g., informative biological indexes for things including brain state (e.g., attention, arousal, mood, etc.), physical state (fatigue, exertion, etc.), wellbeing (blood pressure, immune system status, hydration) and emotional feelings (anger, desire, disgust, etc.)). In some applications, stimulations devices can alternatively be required to be positioned sufficiently far away from other anatomic targets on the body at which energy delivery could cause damage or adverse effects to physiological stage or at which signals are present that obscure or interfere with the targeted recording signals. Further, it may be beneficial to have devices for recording and/or stimulating multiple locations on one or more targets.

Methods of positioning wearable device on a body of a user, with accurate positioning relative to anatomical points of interest, can improve the function of stimulation devices. For example, once placement of a stimulation device is initially calibrated, an end user (i.e., a subject or patient) can accurately and reliably continue to use the stimulation device. Since a majority of end users do not have medical training, methods and systems for optimal positioning of stimulation devices can produce better outcomes for patients.

Methods and systems of placing a stimulation device over an anatomic target (e.g., vagus or other peripheral nerve) are provided. Such methods could be used for locating peripheral nerves in general for other applications.

In some instances, a cardboard or paper guide can be provided around an adhesive electrode patch. Excess material around the edges can be shaped in a such a way that would allow the user to align points of the guide to aspects of their neck/jawline to align with a peripheral nerve (e.g., vagus nerve). When these points are lined up, the adhesive electrode patch can be optimally positioned and the adhesive portion can be adhered to the location by, for example, applying pressure. The guide could have a perforated edge between itself and the patch, allowing removal of excess adhesive portion, leaving only the adhesive patch correctly placed.

In this aspect, the adhesive guide can be configured for an individual patient by, for example, taking a photograph of the general location and supplying the photograph to a system capable of generating dimensions for an individual patient.

In some instances, imaging (e.g., photograph, MM, CAT, PET or other suitable imaging methodology) can be performed on a neck of patient to locate the nerve and have a small tattoo or a non-visible marker placed over the optimal location (i.e., an alignment marker) for placement of a nerve stimulation device. Alternatively, imaging can be used to locate an optimal location on a peripheral nerve, and a reference picture can be provided to a healthcare professional or a patient showing an optimal placement location. In some instances, the alignment marker can be added as a guide by a healthcare professional based in visual inspection or imaging. The patient can then use the alignment marker as a guide for placement of the stimulation device.

In another aspect, an application for phone or computer can use a imaging device to scan the user's body (e.g., face or neck), and then overlay an augmented reality (AR) diagram or cartoon rendering of the nerve location on a body portion (e.g., neck). Three-dimensional imaging data sets of human subjects (e.g., MM) could be used to generate a model of the location of a nerve, such as the vagus nerve, based on the external body structure above the nerve. For example, to predict the location of the vagus nerve, measurements of the neck and other facial and shoulder structures via imaging could be fed into the model which would then determine the most likely location of the vagus nerve relative to specific landmarks. This model could then be used to tailor the overlaid AR diagram to each user, increasing accuracy.

Many nerves are located next to blood vessels. For example, the vagus nerve runs in the same sheath as the carotid artery, and the carotid artery produces a rhythmic signal that can be measured on the skin (e.g., via EKG or skin deformation). Devices and systems described herein can locate the point nearest to a location above the carotid artery, or other target blood vessel, through measuring signals produced by the pumping of blood through the vessel (e.g., having the loudest rhythmic signal measured by a microphone, flat electrode, or other device or sensor placed on the surface).

The location of the vagus nerve is well-defined relative to certain muscles in the neck. EMG activity could be used to identify the location of these muscles and then triangulate the position of the vagus nerve. A user could be instructed to move their head in certain motions (e.g., look left, look up, etc.) during the calibration process to facilitate EMG measurement.

An oversized electrode patch containing a grid of electrodes can be used. This exemplary grid electrode device could apply test stimulation across different electrode pairs (i.e., different locations). In this aspect, the user could place the grid electrode device on their neck in the general location for stimulus application. Feedback provided by the electrodes or other sensors in the patch can be used to locate a peripheral nerve (e.g., vagus nerve), potentially using one of the above-mentioned methods, and identify which electrodes in the grid are located nearest to it.

Alternatively, identification of the optimal electrodes for stimulation (i.e., electrodes nearest the nerve) within the grid electrode device can be performed using closed-loop feedback in response to stimulation. A system with multiple electrodes in a patch could test stimulating across different pairs of electrodes while measuring the neuromodulation induced via a variety of noninvasive indexes to gauge which pair of electrodes induce the strongest neuromodulation. For example, a calibration sequence can be used at startup of a nerve stimulation device to intelligently test various combination of electrodes pairs, measure the response to stimulation for each pair, and then determine the pair in the grid that would produce the strongest response.

Indexes of activation of target neural circuitry, which provide feedback that can be measured in response to stimulation with parameters or locations includes, but is not limited to, pupil dilation/constriction, EEG response (e.g., change in synchronization or change in ratio of power bands), EKG rate or variability, respiratory rate or variability, perspiration, EMG signals, performance on perceptual/cognitive/memory tests, or change in blood pressure.

Methods of positioning a nerve stimulation device on a body of subject by aligning the nerve stimulation device proximal to at least a first location on a peripheral nerve of the body of the subject are provided. Once the device is placed, peripheral nerve stimulation can be applied to the subject. In some instances, the peripheral nerve is a vagus nerve.

In some instances, the aligning of the nerve stimulation device is performed using an alignment device selected from the group consisting of a geometric guide, an imaging device, an auditory device, an electrical signal detection device, and an electrode grid.

In some instances, the alignment device detects a measured response (e.g., EKG (electrocardiography), EMG (electromyogram), EEG (electroencephalography), pupil dilation, perceptual activity, task performance, respiration, perspiration, and blood pressure.

In some instances, at least one location is selected from the group consisting of a neck, a jaw, a shoulder, and an ear of the body of the subject.

In some instances, nerve stimulation (e.g., tonic vagus nerve stimulation) is applied to the at least a first location.

In some instances, nerve stimulation (e.g., tonic vagus nerve stimulation) is applied to at least a second location on a peripheral nerve. In some instances, a distance between the first location and the second location is about 1 to 10 cm. In some instances, the tonic vagus nerve stimulation is applied from about 3 to about 60 seconds.

Aspects described herein provide systems for positioning a nerve stimulation device on a body of a subject, comprising an alignment device (e.g., geometric guide, an imaging device, an auditory device, an electrical signal detection device, and an electrode grid) configured to identify at least one optimal location on a peripheral nerve of the body of the subject to apply peripheral nerve stimulation.

In some instances, the peripheral nerve is a vagus nerve.

In some instances, the alignment device detects a measured response (e.g., EKG, EMG, EEG, pupil dilation, perceptual activity, and blood pressure).

In some instances, the at least one location is selected from the group consisting of a neck, a jaw, a shoulder, and an ear of the body of the subject.

In one aspect, the nerve stimulation (e.g., tonic vagus nerve stimulation) is applied to the at least a first location. In some instances, the nerve stimulation (e.g., tonic vagus nerve stimulation) is applied to the at least a second location.

In one aspect, a distance between the first location and the second location is about 1 to 10 cm.

In some instances, the nerve stimulation (e.g., tonic vagus nerve stimulation) can be applied from about 3 to about 60 seconds.

In a further aspect, the systems and methods disclosed herein can be used to identify a nerve location in order to avoid placing a device at the nerve location. In some instances, a patient or healthcare professional may wish to avoid stimulating a particular nerve or group of nerves in order, for example, to focus a treatment on a different nerve or group of nerves. Aspects described herein provide methods of positioning a nerve stimulation device on a body of subject by aligning the nerve stimulation device proximal to at least a first location on a nerve of the body of the subject, wherein nerve stimulation is not applied to the at least a first location.

In some instances, a first method of positioning a stimulation device on an anatomic target of a subject is provided. The first method comprises positioning an alignment guide adjacent to at least a first alignment point associated with the anatomic target on the subject, wherein the alignment guide indicates a first target location on the anatomic target of the subject, and positioning the stimulation device at the first target location.

The term “anatomic target” refers to a region or location on an animal or human body (e.g., jawline, collarbone, neck, torso, back, arm, leg, and foot). An anatomic target can refer to a general location where a target of interest is located. For example, a peripheral nerve of interest can be located within an anatomic target such as the neck.

The term “alignment guide” refers to a device (or a digital representation of a region on a body) that guides a user in more precise placement of a stimulation device on a target location within an anatomic target. For example, the alignment guide can be shaped to fit within an anatomic target (e.g., neck) and guide a user regarding more precise placement of a stimulation device on a target (e.g., peripheral nerve). The dimensions and shape of an alignment guide can be based on general anatomy of an animal or human or on a scan or image that is taken of anatomic target.

The term “at least a first alignment point” refers to one or more location within or proximal to anatomic target for initial placement of the alignment guide. When an alignment guide is positioned at an alignment point, the alignment guide is positioned to identify a target location to the user. For example, if a user positions the alignment guide at an alignment point located at a position on the collarbone closest to the neck, the alignment guide can indicate a target location 3 cm towards the arm. It is understood that two or more alignment points can be used for additional guidance when positioning the alignment guide.

The term “target location” refers to a specific anatomic location where stimulation with the stimulation is to be applied (e.g., peripheral nerve, muscle group, etc.). In some instances, a stimulation device utilizes at least two contact points—one positive electrode and one negative electrode. In order to generate current to run into the body, these electrodes are insulated from one another except for the path of conductance through the skin. In some instances, a target location can encompass the area where both the positive and negative electrodes are placed. In some instances, a target location for one electrode can be identified and a target location for at least a second electrode can be identified.

The term “stimulation device” refers to a device capable of generating arousal-linked neuromodulation of sensory processing, for example, through peripheral stimulation of the vagus nerve. Stimulation devices described herein can be externally worn, transcutaneous vagus nerve stimulators (nVNS). In some aspects, stimulation devices are lightweight, noninvasive neural interface that can be easily taken on and off, allowing users to engage the device during important moments. For example, nVNS can be used during social situations where ability to communicate clearly is key or when working in potentially dangerous conditions or with potentially dangerous equipment.

In some instances, the stimulation device can provide tonic vagus nerve stimulation to the subject wherein the sensory processing of the subject is modified. The term “tonic” refers to sustained or graded stimulation or a sufficiently rapid duty cycle stimulation. See, e.g., WO2020252428.

In some instances of the first method, stimulation (e.g., peripheral nerve electrical stimulation) is applied to the first target location with the stimulation device. Some instances of the first method further comprise detecting a measured response of the subject to the stimulation. The term “measured response” refers to a biological response of a subject to stimulation that can be quantified. For example, the measured response can be selected from the group consisting of one or more of EKG (electrocardiogram), EMG (electromyogram), EEG (electroencephalogram), pupil dilation, task performance, respiration, perspiration, perceptual activity, skin conductance, and blood pressure.

In some instances of the first method, the alignment guide can detect the measured response. Some instances of the first method further comprise adjusting the position of the stimulation device based on the measured response. For example, if the measured response is pupil dilation, and the degree of pupil dilation following application of stimulation is suboptimal, the position of the stimulation device can be moved, and pupil dilation can be measured again following stimulation to determine if the new position of the stimulation device results in more optimal pupil dilation.

In some instances of the first method, the anatomic target is a peripheral nerve. In some instances of the first method, the peripheral nerve is a vagus nerve.

In some instances of the first method, the stimulation applied to the first target location is tonic electrical stimulation. The tonic electrical stimulation can be applied for at least 3 seconds or can be applied up to 8 hours.

In some instances of the first method, the anatomic target is a muscle group.

In some instances of the first method, when the alignment guide is positioned proximal to the at least a first alignment point, the alignment guide indicates at least a second target location on the anatomic target. In some instances, when the alignment guide is positioned proximal to at least a second alignment point, the alignment guide indicates at least a second target location on the anatomic target. It is understood that the alignment guide can be positioned proximal to one or more alignment points on the anatomic target. Alternatively, positioning the alignment guide proximal to a first alignment point can indicate one or more target locations on the anatomic target.

In some instances of the first method, the alignment guide is printed using a 2D printer or a 3D printer. The 2D or 3D printer can be programmed to print an alignment guide based on a scan of the anatomic target or target location and substantially (e.g., greater than 50%) conform to the contours and dimensions of the anatomic target or target location to produce a custom fit alignment guide.

In some instances of the first method, the stimulation device is printed using a 2D printer or a 3D printer and can substantially conform to and align with the first target location. The 2D or 3D printer can be programmed to print stimulation device based on a scan of the anatomic target or target location and substantially (e.g., greater than 50%) conform to the contours and dimensions of the anatomic target or target location to produce a custom fit stimulation device.

In some instances of the first method, a distance between the first target location and the second target location is about 1 to about 10 cm. Alternatively, the distance between the first target location and the second target location is about 2 to about 8 cm, about 3 to about 7 cm, about 4 to about 6 cm, or about 5 cm.

Some instances of the first method further comprises applying stimulation to the anatomic target at the second target location.

In some instances of the first method, the alignment guide is selected from the group consisting of a geometric guide, an imaging device, an auditory device, an electrical signal detection device, and an electrode grid.

In some instances of the first method, the stimulation device comprises an electrode grid. The electrode grid can comprise a plurality of electrodes. In some instances, one or more of the plurality of electrodes applies a stimulation to the anatomic target and a biological response to the stimulation is measured. In some instances of the first method, one or more of the plurality of electrodes is selected to apply the stimulation based on the biological response.

Some instances of the first method, further comprises adjusting the position of the stimulation device based on the biological response.

In some instances, a second method of positioning a stimulation device on an anatomic target of a subject is provided. The second method comprises scanning the anatomic target on a body of a subject with an imaging device to obtain a target location scan for a target location, comparing the target location scan to one or more known scans of the anatomic target, generating an alignment guide for the anatomic target based on a comparison of the target location scan to the one or more known scans, positioning the alignment guide at a first alignment point wherein the alignment guide indicates a first target location on the anatomic target, and positioning the stimulation device at the first target location.

Scans of an anatomic location can be obtain by a variety of methods (e.g., magnetic resonance imaging (MM), computed tomography (CT) etc.) or by using a camera, including a camera built into a portable device such as a phone. The term “known scans” refers to one or more previously obtained scans of an anatomic region (e.g., neck, back, head, arm, etc.) that can be used as a point of reference to scans taken of a particular subject. The known scans can be stored in any suitable computer storage medium, including a cloud drive or similar networked device. The known scans can be of other subjects, or they can be scans of a particular subject that have been previously obtained. A “known scan” can also include a real-time video feed or use of augmented reality (AR) and an AR device to visualize placement of a stimulation device.

In some instances of the second method, the alignment guide is digitally generated from the target location scan, overlaid on the target location scan, and displayed to a user in real-time. The alignment guide can, for example, be overlaid on the target location scan is displayed via light projection. In this aspect, a subject or healthcare provider can visualize a target location in real-time by displaying the alignment guide directed on the anatomic target of the subject.

Some instances of the second method further comprise applying a stimulation to the anatomic target at the first target location with the stimulation device.

The anatomic target can be a peripheral nerve and the peripheral nerve can be a vagus nerve. The stimulation can be tonic electrical stimulation. The tonic electrical stimulation can be applied for at least 3 seconds or for up to 8 hours.

Some instances of the second method comprise applying a stimulation to an anatomic target at a location other than the first target location. In this example, the alignment guide can be used to identify a target location to be avoided when applying stimulation.

In some instances of the second method, the anatomic target is a muscle group.

In some instances of the second method, the alignment guide is printed using a 2D printer or a 3D printer. The 2D or 3D printer can be programmed to print an alignment guide based on a scan of the anatomic target or target location and substantially (e.g., greater than 50%) conform to the contours and dimensions of the anatomic target or target location to produce a custom fit alignment guide.

In some instances of the second method, the stimulation device is printed using a 2D printer or a 3D printer and can substantially conform to and align with the first target location. The 2D or 3D printer can be programmed to print stimulation device based on a scan of the anatomic target or target location and substantially (e.g., greater than 50%) conform to the contours and dimensions of the anatomic target or target location to produce a custom fit stimulation device.

In some instances, a third method of positioning a stimulation device on an anatomic target of a subject is provided. The third method comprises scanning an anatomic target on a body of the subject with an imaging device to obtain a target location scan, generating an alignment guide for the anatomic target based on the target location scan, positioning the alignment guide adjacent to at least a first alignment point, wherein the alignment guide indicates a first target location on the anatomic target of the subject, and positioning the stimulation device at the first target location.

Some instances of the third method further comprise applying stimulation proximal to the first target location.

In some instances of the third method, the alignment guide comprises at least one alignment marker selected from the group consisting of a UV marker, an RFID tag, a radiolabeled chemical, a tattoo, an ink mark, and a jewelry piercing.

Some instances of the third method further comprise applying stimulation proximal to the at least one alignment point.

The anatomic target can be a peripheral nerve. The peripheral nerve can be a vagus nerve.

In some instances of the third method, tonic electrical stimulation is applied to the vagus nerve. The tonic electrical stimulation can be applied for at least 3 seconds or up to 8 hours.

In some instances of the third method, the anatomic target is a muscle group.

In some instances, a fourth method of applying stimulation to an anatomic target of a subject is provided. The fourth method comprises positioning an alignment guide adjacent to at least a first alignment point associated with the anatomic target on the subject wherein the alignment guide indicates a first target location on the anatomic target of the subject, positioning a stimulation device at the first target location, and applying stimulation to a location other than the first target location with the stimulation device.

Aspects described herein provide a system for applying stimulation to an anatomic target on a body of a subject comprising an alignment guide, wherein when the alignment guide is positioned at a first alignment point, the alignment guide indicates a first target location at the anatomic target; and a stimulation device for applying stimulation at the first target location.

In some aspects, the anatomic target is a peripheral nerve. The peripheral nerve can be a vagus nerve.

In some aspects, the stimulation device can apply tonic electrical stimulation to the vagus nerve. In some aspects, the anatomic target is a muscle group. When the alignment guide is positioned proximal to the first alignment point, the alignment guide can indicate at least a second target location on the anatomic target. When the alignment guide is positioned proximal to at least a second alignment point, the alignment guide can indicate at least a second target location on the anatomic target.

In some aspects, the alignment guide is selected from the group consisting of a geometric guide, an imaging device, an auditory device, an electrical signal detection device, and an electrode grid.

In some aspects, the alignment guide can detect a measured response (e.g., EKG, EMG, EEG, pupil dilation, task performance, respiration, perspiration, perceptual activity, skin conductance, and blood pressure).

The system can further comprise a measured response detector (e.g., a wrist-worn monitor, smart glasses, electrodes, camera, imaging device, heart monitor, tablet, cell phone, functional magnetic resonance imaging device (fMRI), computer, motion tracking system, and an electroencephalography (EEG) headcap).

The measured response can be selected from the group consisting of EKG, EMG, EEG, pupil dilation, task performance, respiration, perspiration, perceptual activity, and blood pressure.

In some aspects, the system further comprises an imaging device for scanning an anatomic target on a body of the subject.

In some aspects, the system further comprises a database comprising one or more known scans for the anatomic target.

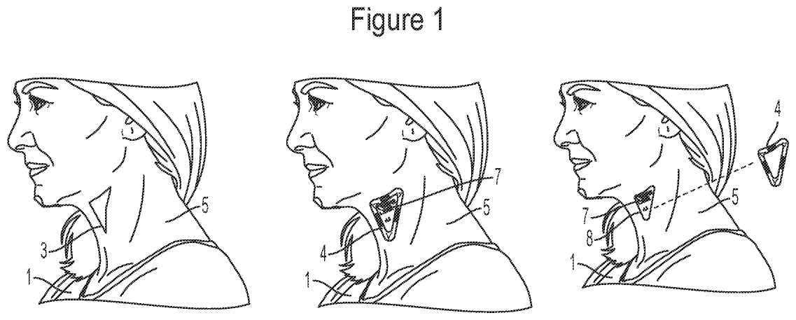

FIG. 1 illustrates an exemplary method of placing an alignment guide at an anatomic location on a subject to guide positioning of a stimulation device on the subject. As shown in FIG. 1, subject 1 having neck 5 as an anatomic target 5 has an alignment point 3 identified on neck 5 (left panel). Alignment guide 4 is shown around the perimeter of stimulation device 7 and is positioning at alignment point 3 (middle panel). After positioning at alignment point 3, alignment guide 4 is removed leaving stimulation device 7 positioned at the target location 8 on neck 5 (right panel). It is understood that stimulation device 7 can alternatively initially be separate from alignment guide 4 and that stimulation device 7 and alignment guide 4 can have similar or different dimensions or shapes.

In some instances, physical exterior features (e.g., muscles, ridges, bones, color changes) define a target location. For example, transcutaneous cervical vagus nerve stimulation (tcVNS) is targeted where the vagus nerve runs parallel and proximal to the carotid artery. On the neck (i.e., cervical location), there is an area known as the carotid triangle whose exterior is defined posteriorly by the sternocleidomastoid muscle, anteroinferiorly by the omohyoid muscle, and superiorly by the digastric muscle. This creates a muscle triangle outline of raised muscles in the neck (alignment point 3 outlined in red in FIG. 1) that can be located by gently palpating the area with fingertips.

As shown in FIG. 1, alignment guide 4 can be shaped such that its edges align with the above described muscle triangle outline, and stimulation device 7 can be shaped to fit within alignment guide 4. The shape of alignment guide 4 and stimulation device 7 can be customized to a particular subject based on imaging performed on the subject.

In use, the subject can align the exterior edges of alignment guide 4 with the edges of an anatomical features (e.g., the muscle triangle outline) resulting in stimulation device 7 being aligned with the vagus nerve running next to the carotid artery.

In some instances, alignment guide 4 may be removed, once stimulation device 7 is positioned and successfully applied, to reduce device size/weight when worn. This feature can be facilitated by an easy to break connection between the device and edge guide (e.g., perforation of material, magnet).

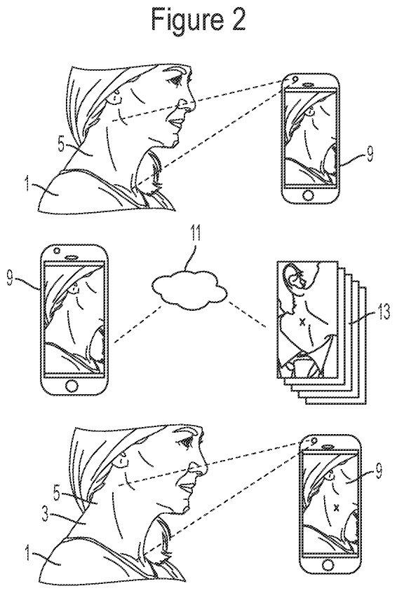

As shown in FIG. 2, imaging device 9 can be used to take an image or scan of anatomic target 5 on subject 1 (top panel). The image displayed on imaging device 9 can be uploaded to cloud storage 11 and compared to known scan 13 (optionally stored in cloud storage 11) of a similar anatomic target (middle panel). Known scan 13 can be overlayed on the scan of anatomic target 5 on imaging device 9 and used as an alignment guide to place a stimulation device (not shown) at alignment point 3 on anatomic target 5 (bottom panel).

The scanned image is then compared with a database of images with known correct labeling of the target location (e.g., red x) either via the cloud or locally on a device. Based on the comparison, an estimate of where the target location is on the newly scanned image can be created.

In some instances, the user can then be shown in real-time the target location digitally overlaid on their body (e.g., red x) on the device they used to scan their body (e.g., cell phone or tablet with a display and built-in camera).

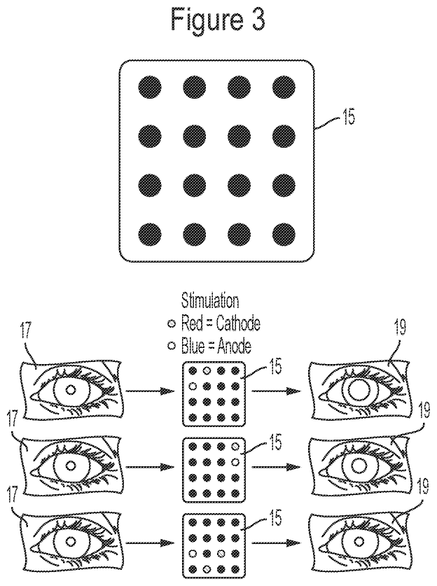

FIG. 3 (top panel) shows exemplary electrode grid 15 having a 4×4 grid of individual electrodes. Each electrode can provide stimulation as described herein. FIG. 3 (bottom panel) illustrates measuring first pupil dilation 17 before and second pupil dilation 19 after stimulation of varying pairs of electrodes (red=cathode, blue=anode). A user or healthcare professional can identify the optimal electrode pair stimulation that produces desired second pupil dilation 19. In this manner, electrode grid 15 can be used as an alignment guide to identify optimal stimulation of one or more electrodes to provide a desired measured response (e.g., pupil dilation) following stimulation at a target location.

In some instances, a stimulation device can have a plurality of interface points (e.g., a neurostimulation patch having a grid of electrode contacts points). The grid can be large enough to cover an anatomic target area such that a specific target location would be located within the anatomic area covered by the grid. Turning specific electrodes on and off within the grid followed by measuring a biological response can be used to identify an optimal target location as illustrated in FIG. 3.

### REFERENCES

1. Lu, L., et al. Wearable Health Devices in Health Care: Narrative Systematic Review. JMIR Mhealth Uhealth 8, e18907 (2020).
2. Johnson, K. T. & Picard, R. W. Advancing Neuroscience through Wearable Devices. Neuron 108, 8-12 (2020).
3. Stavropoulos, T. G., Papastergiou, A., Mpaltadoros, L., Nikolopoulos, S. & Kompatsiaris, I. IoT Wearable Sensors and Devices in Elderly Care: A Literature Review. Sensors (Basel) 20 (2020).
4. Mogilner, A. Y. Peripheral Nerve Stimulation for Facial Pain Using Conventional Devices: Technique and Complication Avoidance. Prog Neurol Surg 35, 68-74 (2020).
5. Vaughn, B. V., et al. Intraoperative methods for confirmation of correct placement of the vagus nerve stimulator. Epileptic Disord 3, 75-78 (2001).
6. Mazzola, A. & Spinner, D. Ultrasound-Guided Peripheral Nerve Stimulation for Shoulder Pain: Anatomic Review and Assessment of the Current Clinical Evidence. Pain Physician 23, E461-e474 (2020).
7. Dunn, J., Runge, R. & Snyder, M. Wearables and the medical revolution. Per Med 15, 429-448 (2018).
8. Temel, Y., et al. Neuromodulation in psychiatric disorders. Int Rev Neurobiol 107, 283-314 (2012).
9. Günter, C., Delbeke, J. & Ortiz-Catalan, M. Safety of long-term electrical peripheral nerve stimulation: review of the state of the art. J Neuroeng Rehabil 16, 13 (2019).
10. Cotero, V., et al. Peripheral Focused Ultrasound Neuromodulation (pFUS). J Neurosci Methods 341, 108721 (2020).
11. Kubiak, C. A., Kung, T. A., Brown, D. L., Cederna, P. S. & Kemp, S. W. P. State-of-the-Art Techniques in Treating Peripheral Nerve Injury. Plast Reconstr Surg 141, 702-710 (2018).
12. Anderson, H. E. & Weir, R. F. F. On the development of optical peripheral nerve interfaces. Neural Regen Res 14, 425-436 (2019).
13. Rodenkirch, C. & Wang, Q. Rapid and transient enhancement of thalamic information transmission induced by vagus nerve stimulation. J Neural Eng 17, 026027 (2020).
14. Engineer, N. D., et al. Reversing pathological neural activity using targeted plasticity. Nature 470, 101-104 (2011).
15. Tassorelli, C., et al. Noninvasive vagus nerve stimulation as acute therapy for migraine: The randomized PRESTO study. Neurology 91, e364-e373 (2018).
16. Li, R. T., et al. Wearable Performance Devices in Sports Medicine. Sports Health 8, 74-78 (2016).
17. Joshi, M., et al. Wearable sensors to improve detection of patient deterioration. Expert Rev Med Devices 16, 145-154 (2019).
18. Majumder, S., Mondal, T. & Deen, M. J. Wearable Sensors for Remote Health Monitoring. Sensors (Basel) 17 (2017).
19. Sana, F., et al. Wearable Devices for Ambulatory Cardiac Monitoring: JACC State-of-the-Art Review. J Am Coll Cardiol 75, 1582-1592 (2020).
20. Rodenkirch, C., Liu, Y., Schriver, B. J. & Wang, Q. Locus coeruleus activation enhances thalamic feature selectivity via norepinephrine regulation of intrathalamic circuit dynamics. Nature Neuroscience 22, 120-133 (2019).
21. Aston-Jones, G. & Cohen, J. D. An integrative theory of locus coeruleus-norepinephrine function: adaptive gain and optimal performance. Annual Review of Neuroscience 28, 403-450 (2005).
22. Sara, S. J. The locus coeruleus and noradrenergic modulation of cognition. Nature Reviews Neuroscience 10, 211-223 (2009).
23. Joshi, S., Li, Y., Kalwani, Rishi M. & Gold, Joshua I. Relationships between Pupil Diameter and Neuronal Activity in the Locus Coeruleus, Colliculi, and Cingulate Cortex. Neuron 89, 221-234 (2016).
24. Günter, C., Delbeke, J. & Ortiz-Catalan, M. Safety of long-term electrical peripheral nerve stimulation: review of the state of the art. J Neuroeng Rehabil 16, 13-13 (2019).
25. Hulsey, D. R., et al. Parametric characterization of neural activity in the locus coeruleus in response to vagus nerve stimulation. Experimental neurology 289, 21-30 (2017).
26. Conway, C. R., Udaiyar, A. & Schachter, S. C. Neurostimulation for depression in epilepsy. Epilepsy & behavior: E&B 88S, 25-32 (2018).
27. Engineer, N. D., et al. Targeted Vagus Nerve Stimulation for Rehabilitation After Stroke. Frontiers in neuroscience 13, 280 (2019).
28. Peter, N. & Kleinjung, T. Neuromodulation for tinnitus treatment: an overview of invasive and non-invasive techniques. Journal of Zhejiang University. Science. B 20, 116-130 (2019).
29. Spindler, P., Bohlmann, K., Straub, H. B., Vajkoczy, P. & Schneider, U. C. Effects of vagus nerve stimulation on symptoms of depression in patients with difficult-to-treat epilepsy. Seizure 69, 77-79 (2019).
30. van Hoorn, A., et al. Neuromodulation of autism spectrum disorders using vagal nerve stimulation. Journal of clinical neuroscience: official journal of the Neurosurgical Society of Australasia 63, 8-12 (2019).
31. Yang, J. & Phi, J. H. The Present and Future of Vagus Nerve Stimulation. Journal of Korean Neurosurgical Society 62, 344-352 (2019).
32. Lamb, D. G., Porges, E. C., Lewis, G. F. & Williamson, J. B. Non-invasive Vagal Nerve Stimulation Effects on Hyperarousal and Autonomic State in Patients with Posttraumatic Stress Disorder and History of Mild Traumatic Brain Injury: Preliminary Evidence. Frontiers in medicine 4, 124 (2017).
33. Mwamburi, M., Liebler, E. J. & Tenaglia, A. T. Review of non-invasive vagus nerve stimulation (gammaCore): efficacy, safety, potential impact on comorbidities, and economic burden for episodic and chronic cluster headache. The American journal of managed care 23, S317-S325 (2017).
34. Reuter, U., McClure, C., Liebler, E. & Pozo-Rosich, P. Non-invasive neuromodulation for migraine and cluster headache: a systematic review of clinical trials. Journal of neurology, neurosurgery, and psychiatry (2019).
35. Hamer, H. M. & Bauer, S. Lessons learned from transcutaneous vagus nerve stimulation (tVNS). Epilepsy research 153, 83-84 (2019).
36. Mourdoukoutas, A. P., Truong, D. Q., Adair, D. K., Simon, B. J. & Bikson, M. High-Resolution Multi-Scale Computational Model for Non-Invasive Cervical Vagus Nerve Stimulation. Neuromodulation: journal of the International Neuromodulation Society 21, 261-268 (2018).
37. Hulsey, D. R., et al. Reorganization of Motor Cortex by Vagus Nerve Stimulation Requires Cholinergic Innervation. Brain stimulation 9, 174-181 (2016).
38. Hays, S. A., Rennaker, R. L. & Kilgard, M. P. Targeting plasticity with vagus nerve stimulation to treat neurological disease. Prog Brain Res 207, 275-299 (2013).
39. Buell, E. P., et al. Vagus Nerve Stimulation Rate and Duration Determine whether Sensory Pairing Produces Neural Plasticity. Neuroscience 406, 290-299 (2019).
40. Liu, Y., Rodenkirch, C., Moskowitz, N., Schriver, B. & Wang, Q. Dynamic Lateralization of Pupil Dilation Evoked by Locus Coeruleus Activation Results from Sympathetic, Not Parasympathetic, Contributions. Cell Reports 20, 3099-3112 (2017).
41. Correia, C., et al. Global Sensory Impairment in Older Adults in the United States. J Am Geriatr Soc 64, 306-313 (2016).
42. Heine, C. & Browning, C. J. Communication and psychosocial consequences of sensory loss in older adults: overview and rehabilitation directions. Disabil Rehabil 24, 763-773 (2002).
43. Serafini, G., et al. Extreme sensory processing patterns show a complex association with depression, and impulsivity, alexithymia, and hopelessness. Journal of affective disorders 210, 249-257 (2017).
44. Shimizu, V. T., Bueno, O. F. & Miranda, M. C. Sensory processing abilities of children with ADHD. Brazilian journal of physical therapy 18, 343-352 (2014).
45. Carron, S. F., Alwis, D. S. & Raj an, R. Traumatic Brain Injury and Neuronal Functionality Changes in Sensory Cortex. Frontiers in systems neuroscience 10, 47 (2016).
46. Al Saif, A. A. & Al Senany, S. Determine the effect of neck muscle fatigue on dynamic visual acuity in healthy young adults. J Phys Ther Sci 27, 259-263 (2015).
47. Anton-Erxleben, K. & Carrasco, M. Attentional enhancement of spatial resolution: linking behavioural and neurophysiological evidence. Nature reviews. Neuroscience 14, 188-200 (2013).
48. Tseng, Y. C., Liu, S. H., Lou, M. F. & Huang, G. S. Quality of life in older adults with sensory impairments: a systematic review. Qual Life Res 27, 1957-1971 (2018).
49. Nowak, D. A. & Hermsdorfer, J. Selective deficits of grip force control during object manipulation in patients with reduced sensibility of the grasping digits. Neurosci Res 47, 65-72 (2003).
50. Reed-Jones, R. J., et al. Vision and falls: A multidisciplinary review of the contributions of visual impairment to falls among older adults. Maturitas 75, 22-28 (2013).
51. Hennissen, L., et al. Cardiovascular Effects of Stimulant and Non-Stimulant Medication for Children and Adolescents with ADHD: A Systematic Review and Meta-Analysis of Trials of Methylphenidate, Amphetamines and Atomoxetine. CNS Drugs 31, 199-215 (2017).
52. Spiller, H. A., Hays, H. L. & Aleguas, A., Jr. Overdose of drugs for attention-deficit hyperactivity disorder: clinical presentation, mechanisms of toxicity, and management. CNS drugs 27, 531-543 (2013).
53. Dubljevic, V. Prohibition or coffee shops: regulation of amphetamine and methylphenidate for enhancement use by healthy adults. Am J Bioeth 13, 23-33 (2013).
54. Talih, F. & Ajaltouni, J. Probable Nootropicinduced Psychiatric Adverse Effects: A Series of Four Cases. Innovations in clinical neuroscience 12, 21-25 (2015).
55. Gualtieri, F. Unifi nootropics from the lab to the web: a story of academic (and industrial) shortcomings. Journal of enzyme inhibition and medicinal chemistry 31, 187-194 (2016).
56. Mesin, L. Crosstalk in surface electromyogram: literature review and some insights. Phys Eng Sci Med 43, 481-492 (2020).

1.-63. (canceled)

64. A system for generating arousal-linked neuromodulation of sensory processing, the system comprising:
a transcutaneous vagus nerve stimulator configured to generate arousal-linked neuromodulation of sensory processing, wherein the transcutaneous vagus nerve stimulator includes at least two electrodes; and
an alignment guide configured to guide a user to position the transcutaneous vagus nerve stimulator over a subject's vagus nerve.

65. The system of claim 64, wherein the alignment guide is arranged to align with aspects of the subject's neck.

66. The system of claim 64, wherein the alignment guide is arranged to align with at least one muscle of the subject's neck.

67. The system of claim 64, wherein the alignment guide is arranged to align with a triangle whose exterior is defined posteriorly by a sternocleidomastoid muscle, anteroinferiorly by an omohyoid muscle, and superiorly by a digastric muscle.

68. The system of claim 64, wherein the transcutaneous vagus nerve stimulator comprises a grid of electrodes.

69. The system of claim 68, wherein feedback provided by the electrodes is used to locate the subject's vagus nerve.

70. The system of claim 69, wherein the feedback provided by the electrodes represents the pumping of blood through the carotid artery.

71. The system of claim 64, wherein the transcutaneous vagus nerve stimulator comprises a grid of electrodes, and wherein the transcutaneous vagus nerve stimulator is configured to identify electrodes within the grid that are nearest to the subject's vagus nerve using closed-loop feedback.

72. The system of claim 64, wherein the alignment guide is generated by imaging a portion of the subject's body.

73. The system of claim 64, wherein the alignment guide is configured to be removable from the transcutaneous vagus nerve stimulator after the transcutaneous vagus nerve stimulator has been applied to the subject's body.
